(19) United States
(12) Patent Application Publication
(10) Pub. No.: US 2005/0277141 A1
(43) Pub. Date: Dec. 15, 2005

(54) Process for analyzing a complex biological state of biosystem and support permitting its direct interpretation

(75) Inventors: Vincent Fert, Marseille (FR); Fabienne Hermitte, La Ciotat (FR); Nicolas Maroc, Saint Zacharie (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: IPSOGEN, a corporation of France, Luminy Biotech Entreprise, Marseille Cedex (FR)

(21) Appl. No.: 11/117,113

(22) Filed: Apr. 28, 2005

Related U.S. Application Data
(63) Continuation of application No. PCT/FR03/03185, filed on Oct. 27, 2003.

(30) Foreign Application Priority Data
Oct. 28, 2002 (FR) FR 02/13487

(51) Int. Cl. C12Q 1/68

(52) U.S. Cl. 435/6

(57) ABSTRACT

A process for analyzing a complex biological state of a biosystem from a sample of nucleic acids isolated from the biosystem including hybridization of the nucleic acids with probes fixed on a support, including: (a) contacting marked polynucleotide sequences prepared from nucleic acids of the sample with a first set of specific polynucleotide probes (E1) of a biological state and at least a second set of reference polynucleotide probes (E2) prepared from a molecule of reference nucleic acid for the biological state under conditions permitting the formation of hybridization products, and (b) detecting the hybridization products formed in step (a), wherein the first and second sets (E1, E2) are fixed on a support and the first set of probes includes n groups of probes, each group being characteristic of a different genetic profile that influences the biological state and can therefore alter it, and each group being assembled on the support in separate positions that are also separated from that (those) of the second set (E2) of probes to permit analysis of the genetic profile of the biological state by detecting the position of the hybridization products formed with at least one probe of the first set, relative to the position of hybridization products formed with the second set of probes.

### RELATED APPLICATION

This is a continuation of International Application No. PCT/FR2003/003185, with an international filing date of Oct. 27, 2003 (WO 2004/040015, published May 13, 2004), which is based on French Patent Application No. 02/13487, filed Oct. 28, 2002.

### FIELD OF THE INVENTION

This invention relates to the analysis of complex molecular systems, especially to the diagnosis of a complex biological state of a biosystem and, more particularly to diagnostic processes that realize a molecular hybridization between a molecular probe and a target taken from a sample of this biosystem.

### SUMMARY OF THE INVENTION

This invention relates to a process for analyzing a complex biological state of a biosystem from a sample of nucleic acids isolated from the biosystem including hybridization of the nucleic acids with probes fixed on a support, including: (a) contacting marked polynucleotide sequences prepared from nucleic acids of the sample with a first set of specific polynucleotide probes (E1) of a biological state and at least a second set of reference polynucleotide probes (E2) prepared from a molecule of reference nucleic acid for the biological state under conditions permitting the formation of hybridization products, and (b) detecting the hybridization products formed in step (a), wherein the first and second sets (E1, E2) are fixed on a support and the first set of probes includes n groups of probes, each group being characteristic of a different genetic profile that influences the biological state and can therefore alter it, and each group being assembled on the support in separate positions that are also separated from that (those) of the second set (E2) of probes to permit analysis of the genetic profile of the biological state by detecting the position of the hybridization products formed with at least one probe of the first set, relative to the position of hybridization products formed with the second set of probes.

This invention also relates to a support including a first set of polynucleotide probes (E1) fixed to a surface of the support and at least one second set of polynucleotide probes (E2) arranged according to the process for analyzing a complex biological state of a biosystem.

This invention further relates to a device for reading a support for carrying out the analytical process for analyzing a complex biological state of a biosystem including a fixed element (F) including inscriptions relative to a first set of specific probes (E1) that permit identification on the support of positioning of hybridization products with the specific probes and a cavity with a shape and size similar to those of the support in which cavity the support is arranged by sliding in grooves (R) provided at an end thereof.

This invention still further relates to a process of in-vitro detecting the MLL gene in a patient including performing the process of analyzing a complex biological state of a biosystem, wherein the first set of probes (E1) includes a plurality of probes including at least one probe capable of separately hybridizing a nucleic acid sequence of one or several partner genes selected from the group consisting of AB11; AF10; AF15q14; AF17; 1F1p; AF1q; AF3p21; AF4; AF5q31; AF6; AF6q21; AF9; AFX-1; CPB; CDK6; EEN; EEL; ENL; FBP17; GAS7; GEPHYRIN; GMPS; GRAF; hcdcrel-1; LAF4; LARG; LCX; LPP; MSF; p300; RARA and SEPTIN and the second set of probes (E2) includes a plurality of probes capable of hybridizing all or part of a specific sequence of nucleic acid of the MLL gene, and viewing the results.

### BRIEF DESCRIPTION OF THE DRAWINGS

The invention is illustrated with the aid of examples described in the following and by attached figures.

FIG. 1 shows a scheme of different hybridization reactions produced between the polynucleotide sequences obtained from a sample, here specific probes of partner genes capable of producing a translocation and the reference probes, here specific probes of the MLL gene.

FIG. 2 shows a scheme of a preferred spatial distribution in which the reference probes have been arranged in a configuration in the form of an “L” on the support. The specific probes were arranged according to horizontal alignments with each set of probes arranged on one and the same horizontal line corresponding to a plurality of specific probes of a single partner gene. Moreover, control probes were arranged in a configuration in the form of an inverted “L”.

FIG. 3 shows a scheme of a support in accordance with aspects of the invention on which the emplacements of the different sets of probes are indicated in a precise manner:

E1: specific probes of the translocations;

E2: reference probes.

FIG. 4 is a scheme of a hybridization in accordance with aspects of the invention in which a set of specific probes of marker genes with a good prognostication of breast cancer were assembled to the right of the support and a second set of specific probes of marker genes with a bad prognostication of breast cancer were assembled on the right. A set of control probes was arranged on the left. After revelation of the hybridization the intensity of the signals obtained for each group permits the immediate visual classification of the tumor analyzed.

FIG. 5 illustrates a scheme of a hybridization support in accordance with aspects of the invention in which the assembly of the specific probes of stem cells on the surface of the hybridization support re-creates an image of the regions of the embryonic brain. Here the arrangement reproduces an image of the embryonic brain, e.g., according to John L. R. Rubenstein, MD., Ph.D.J. Am. Acad. Child Adolesc. Psychiatry, 37 (5): 561-562, 1998.

FIG. 6 shows a support for realizing the process of the invention on which a mask is used comprising reference marks in order to further facilitate the identification of the hybridization products formed and the interpretation of the results.

FIG. 7 shows a reading device of the support (S) on which the analytic process of the invention is carried out. This device comprises a fixed element (F) comprising inscriptions relative to the first set of specific probes (E1), e.g., those corresponding to the specific probes that hybridize with all or part of the partner genes and permit the identification of their position on the support. This device comprises a cavity with a shape and size basically similar to those of the support (S) in which cavity the support (S) can be arranged, e.g., by sliding in grooves (R) provided to this end.

FIG. 8 shows a hybridization support in accordance with the 8 concerning the MLL gene in the form of a diagram.

FIG. 9 shows a result of a positive test in accordance with the invention showing the identification of a translocation of the MLL gene with a partner (here AF4).

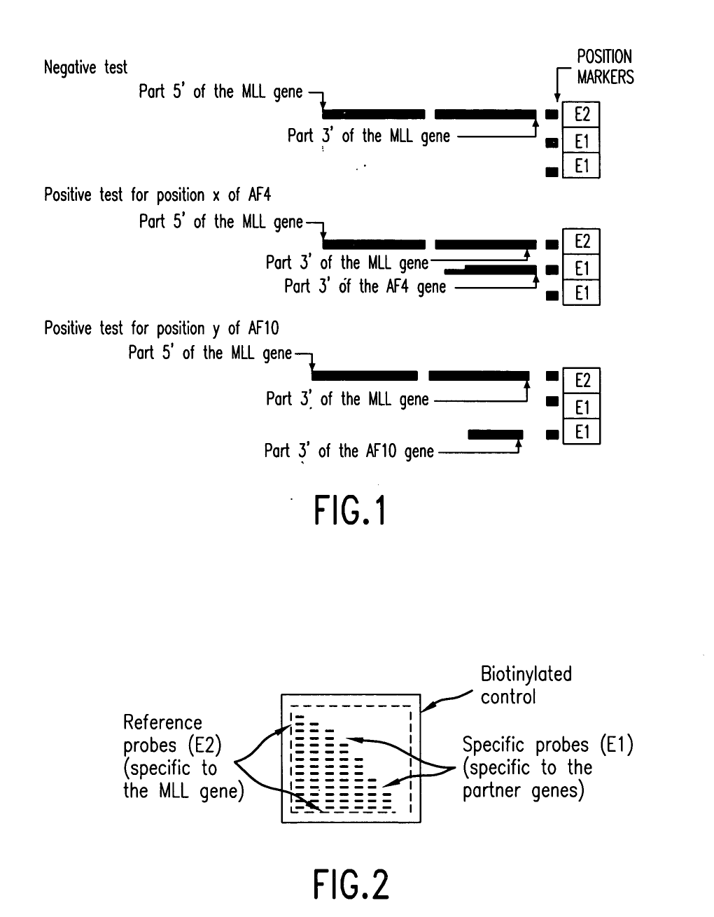

### DETAILED DESCRIPTION

The term ‘complex biological state of a biosystem” denotes within the framework of this invention every state of this biosystem characterized by its genetic profile determined by sets of sequences of its own nucleic acids, which genetic profile can be modified by different biological processes such as introduction of foreign nucleic acids, e.g., as a consequence of bacterial or viral infections or co-infections, or by modification of nucleic acids initially present in the biosystem by processes of cancerization as well as of mutations, deletions or translocations.

These biosystems can also undergo modifications as a result of interactions of the receptor-ligand type that can initiate cascade reactions also resulting in modifications of the initial profile.

The term “biosystem” denotes in the framework of the invention every environment comprising living organisms from which genetic information can be extracted by isolating one or several molecules of nucleic acid of their genome. This can be microorganisms (viruses, algae, bacteria) or higher unicellular or multicellular organisms. These biosystems are present in various environments such as water, animal or vegetable biological fluids and tissues and agro-food preparations.

The methods permitting documentation of linking reactions between a target and a molecular probe (reactions of molecular hybridization) are realized either in solution or on physical supports. In the latter instance, documenting the reaction can take place by a variety of processes with the common goal of producing a signal that can be localized, is quantifiable or non-quantifiable and reflects the phenomenon of linking.

The progress realized in the understanding of biological mechanisms leads to exploitation of systems permitting analysis of simultaneous and numerous reactions of molecular hybridization to obtain new and more precise information (e.g., biochips). Analysis of these reactions requires sophisticated instruments most of the time as well as interpretive software so that the user can correctly exploit the results.

This invention is constituted of an analytical process that involves such numerous and simultaneous reactions of molecular hybridization and is designed to simplify interpretation of the results of such a complex hybridization system, which process comprises preliminarily placing a spatial distribution on a support which distribution is obtained by a particular arrangement of the various probes arranged on the support in such a manner as to produce an image, e.g., a geometric figure, at the end of the hybridization reaction, the interpretation of which image is evident and simplified to become independent of having to use complex analytical processes such as algorithmic methods supplied by software.

More precisely, the invention relates to a process for analyzing a complex biological state of a biosystem from a sample of nucleic acids isolated from the biosystem comprising hybridizing the nucleic acids with probes fixed on a support, comprising the following steps:

(a) contacting marked polynucleotide sequences prepared from nucleic acids of the sample with a first set of specific polynucleotide probes (E1) of a biological state and at least a second set of reference polynucleotide probes (E2) prepared from a molecule of reference nucleic acid for the biological state under conditions permitting formation of hybridization products, and
(b) detecting hybridization products formed in step (a), wherein the first and second sets (E1, E2) are fixed on a support and which first set of probes is constituted of n groups of probes, each group being characteristic of a different genetic profile that influences the biological state and can therefore alter it, and each group being assembled on the support in separate positions that are also separated from that (those) of the second set (E2) of probes in such a manner as to permit analysis of the genetic profile of the biological state by detecting the position of the hybridization products formed with at least one probe of the first set, advantageously relative to the position of a hybridization products formed with the second set of probes.

According to one aspect, the analytical process of the invention relates to analysis of the physiological state of the patient, as described above, from a sample of nucleic acids of this patient in which:

the first set of probes (E1) is a set of polynucleotide probes prepared from specific molecules of nucleic acid of the physiological state and the second set of reference probes (E2) is a set of probes prepared from a molecule of reference nucleic acid for the physiological state under conditions permitting formation of hybridization products.

The polynucleotide sequences of step (a) of the analytical process are preferably marked by any type of marker permitting detection of the position of the hybridization products of step (b). The term “direct detection” denotes obtaining information about the physiological state of the patient without using specific means for interpreting the positioning of the hybridization products. This detection can be made with the naked eye when the markers permit a colorimetric visualization, e.g., biotin, digoxygenine or any other appropriate marker by means of a fluorometric visualization device when the markers are fluorophores, and by means of a radioactive visualization detection device when the markers are emitters of radioactive waves.

Each of the reference polynucleotide probes is advantageously different and prepared from a molecule of nucleic acid corresponding structurally or functionally to the genetic profile of which each of the n groups of probes of the first set is characteristic.

The reference polynucleotide probes are advantageously different and/or identical and prepared from a molecule of reference nucleic acid for the biological state analyzed. Each of the n groups of probes of the first set is preferably arranged in a geometric configuration relative to at least one probe of the second set. Each of the n groups of probes of the first set is preferentially in alignment with or around at least one probe of the second set.

Also, according to another aspect, the probes of the second set are aligned and the probes of each of the n groups of the first set are in alignment with a probe of the first set in such a manner that the n alignments of the probes of the first set are substantially perpendicular to the alignment of the probes of the second set.

The analytical process can also comprise in step (a) implementing a second second set of reference polynucleotide probes. In particular, this second second set of reference polynucleotide probes can be identical to the reference probes of the first set and the probes of the second set are in positions separated from the other probe sets and in a geometric configuration relative to them.

The first set of probes (E1) is preferentially constituted of a plurality of probes comprising at least one probe capable of separately hybridizing a nucleic acid sequence of one or several, e.g., 2 or 3, partner genes constituted of the group: AB11; AF10; AF15q14; AF17; 1F1p; AF1q; AF3p21; AF4; AF5q31; AF6; AF6q21; AF9; AFX-1; CPB; CDK6; EEN; EEL; ENL; FBP17; GAS7; GEPHYRIN; GMPS; GRAF; hcdcrel-1; LAF4; LARG; LCX; LPP; MSF; p300; RARA and SEPTIN and the second set of probes (E2) comprises a plurality of probes capable of hybridizing all or part of a specific sequence of nucleic acid of the MLL gene.

According to one aspect, the probes of the first second set are aligned substantially perpendicularly to the end of the alignment of the probes of the second second set in such a manner as to form a geometric configuration in the shape of an “L”.

According to another aspect, visual detection of the hybridization positions is carried out by means of a mask comprising elements of visual reference marks for each set of probes (E1, E2) and possibly of control probes.

This mask preferably comprises a window in the shape of an “L” that can be superposed on the second set of probes aligned in such a manner as to form a geometric configuration in the form of an “L” on the support and n windows perpendicular to one of the arms of the “L”, each of which n perpendicular windows can be superposed on each of the n alignments of the probes of the first set substantially perpendicular to the alignment of the probes of the second set.

This mask comprises elements of visual reference marking for each set of probes (E1, E2) and can have a geometric configuration reproducing a histological configuration of a tissue or an organ of the system to be analyzed.

The invention also relates to a support for implementing an analytical process as described above comprising, fixed to its surface, a first set of polynucleotide probes (E1) and at least one second set of polynucleotide probes (E2) arranged as previously indicated.

This support can comprise, after implementation of the analytical process, hybridization products of marked polynucleotide sequences on its surface prepared from nucleic acids of the sample with a part of the probes of the first set and all the probes of the second set in such a manner as to form a geometric figure on the surface of this support that can be detected by the naked eye.

This geometric figure advantageously comprises an “L” formed by the hybridization products of marked polynucleotide sequences prepared from nucleic acids of the sample with the probes of the second set.

The invention also relates to a process for direct visualization of a molecular hybridization reaction that realizes the analytical process of the invention followed by visualization of the hybridization product obtained by any known means.

Among the visualization means that can be used, it is possible to cite, e.g., image scanning systems that are used especially when the process of the invention comprises the simultaneous reading of several supports combined with simple software that permit treatment of a limited number of scanned images-objects.

The invention also relates to a device for reading a support for realizing an analytical process of the invention comprising a fixed element (F) comprising inscriptions relative to a first set of specific probes (E1) that permit identification of the positioning on the support of the hybridization products with specific probes and a cavity with a shape and size substantially similar to those of this support in which cavity this support is arranged, advantageously by sliding in grooves (R) provided for this purpose.

The invention also concerns a process for following up a therapeutic treatment of a patient implementing successive analyses of the biological state of the patient by means of an analytical process in accordance with the invention carried out in different stages of this therapeutic treatment.

The invention also concerns the use of a process and/or of a support previously described for the in-vitro detection of the MLL gene in a patient, in particular of a disease implying the MLL gene and at least one of the partner genes of the group described above such as, in particular, a leukemia.

Preferred exemplary embodiments of the analytical process of the invention are described in detail in the following examples.

### EXAMPLE 1

This Example Illustrates An Analytical Process Of The Translocation System Of The MLL Gene.

The MLL gene (mixed lineage leukemia) located on chromosome 11 is rearranged with portion of DNA stemming from a relatively elevated number of distinct genes in a certain number of acute leukemias. The presence of this rearrangement is an independent sign of poor prognis which leads to treating the patient with the particular therapeutic regime. The effectiveness of this therapy can then be evaluated by performing a molecular analysis on the treated patient that permits quantification of the number of residual circulating cells carrying the MLL translocation. This latter analysis can only be realized in an effective manner in the case in which the partner of the MLL gene on the possible thirty ones as well as the precise meeting point between the MLL gene and its partner gene have been precisely identified.

The MLL gene has been identified in the translocations 11q23 and at least 32 partner genes of the MLL gene have been identified. They are illustrated in the following Table 1.

TABLE 1

| No. | Partner | Position | Translocation | Pathological MLL-XX implication |
|---|---|---|---|---|
| 1 | ABI1 | 10p11.2 | t (10; 11) | M4ANLL in an infant |
| | | | (p11.2; q23) | |
| 2 | AF10 | 10p12 | t (10; 11) | Primarily M4/M5 ANLL. ANLL induced by a therapy. Poor |
| | | | (p12; q23) | prognosis. |
| 3 | AF15q14 | 15q14 | t (11; 15) | Acute non-lymphoid leukemia (ANLL) |
| | | | (q23; q14) | |
| 4 | AF17 | 17q21 | t (11; 17) | ANLL and MDS |
| | | | (q23; q21) | |
| 5 | AF1p | 1p32 | t (1; 11) | AMMOL, ALL and ANLL |
| | | | (p32; q23) | |
| 6 | AF1q | 1q21 | t (1; 11) | ANLL essentially M4 |
| | | | (q21; q23) | |
| 7 | AF3p21 | 3p21 | t (3; 11) | ANLL induced by a treatment |
| | | | (p21; q23) | |
| 8 | AF4 | 4q21 | t (4; 11) | Acute leukemia. Typically CD19+ B-ALL, biphenotypic |
| | | | (q21; q23) | AL, sometimes ANLL (M4/M5). Common in infants. |
| | | | variable meeting points | Leukemia induced by a treatment. |
| 9 | AF5q31 | 5q31 | ins (5; 11) | Not well defined, a single case up to the present. |
| | | | (q31; q13q23) | |
| 10 | AF6 | 6q27 | t (6; 11) | Primarily M4/M5 ANLL. ANLL de novo and induced by |
| | | | (q27; q23) | a therapy. Poor prognosis. |
| 11 | AF6q21 | 6q21 | t (6; 11) | ANLL induced by a treatment of the M5 type. |
| | | | (q21; q23) | |
| 12 | AF9 | 9p22 | t (9; 11) | M5/M4 ANLL de novo and induced by a therapy. The |
| | | | (p22; q23) | prognosis can not be as unfavorable as for other leukemias |
| | | | variable meeting points | connected to 11q23 in the de novo cases; very unfavorable |
| | | | on the two genes. | prognosis in the cases of secondary ANLL. |
| 13 | AFX-1 | Xq13 | t (X; 11) | ANLL, T-ALL. Very unfavorable prognosis. |
| | | | (q13q23) | |
| 14 | CBP | 16p13.3 | t (11; 16) | ANLL (t-ANLL) induced by a treatment; can attract close to t |
| | | | (q23; p13) | (11; 22) (q23; q13). Prognosis unfavorable. |
| 15 | CDK6 | 7q21-q22 | t (11; 7) | ALL in the infant |
| | | | (q23; q21) | |
| 16 | EEN | 19p13 | t (11; 19) | Unknown to this day, a single case. |
| | | | (q23; p13) | |
| 17 | ELL | 19p13.1 | t (11.19) | Substantially M4/M5; leukemia induced by a treatment; all |
| | | | (q23: p13.1) | ages. Very unfavorable prognosis. |
| 18 | ENL | 19p13.3 | t (11.19) | ALL (CD19+), biphenotypic AL, ANLL (M4/M5); |
| | | | (q23; p13.3) | primarily congenital; leukemia induced by a treatment. Very |
| | | | | unfavorable prognosis except for the leukemias of the T cells. |
| 19 | FBP17 | 9q34 | t (9; 11) | Unfavorable prognosis. |
| | | | (q34; q23) | |
| 20 | GAS7 | 17p13 | t (11; 17) | Acute non-lymphoid leukemia induced by a treatment with t |
| | | | (q23; p13) | (11; 17) (q23; p13). A single case: a 13-year-old boy with |
| | | | | an M4 ANLL; other cases t (11; 17) (q23; p13) have been |
| | | | | described but the implication of GAS7 has not been proven. |
| 21 | GEPHYRIN | 14q23.3 | t (11; 14) | ANLL and A1 induced by a treatment. |
| | | | (q23; q24) | |
| 22 | GMPS | 3q24 | t (3; 11) | Non-lymphoblastic acute leukemia (M4 ANLL) induced by a |
| | | | (q25: q23) | treatment. |
| 23 | GRAF | 5q31 | t (5; 11) | ANLL. Prognosis unknown, very few cases. |
| | | | (q31; q23) | |
| 24 | hcdcrel-1 | 22q11.2 | t (11; 22) | M4, M2 and M1 ANLL |
| | | | (q23: q11) | |
| 25 | LAF4 | 2q11 | t (2; 11) | |
| | | | (q21; q23) | |
| 26 | LARG | 11q23 | Caryotype normal | Non-lymphocytic acute leukemia (ANLL) with chromosome |
| | | | | 11 apparently normal. Poorly defined: a single case up to the |
| | | | | present. |
| 27 | LCX | 10q22 | t (10; 11) | Adult de novo M2-AML leukemias with trilneage [cannot |
| | | | (q22; q23) | document - probably misspelled -possibly “trilignage” = |
| | | | | “trilineage”] dysplasia. |
| 28 | LPP | 3q28 | t (3; 11) | AML M5 induced by a treatment |
| | | | (q28; q23) | |
| 29 | MSF | 17q25 | t (11; 17) | Leukemia de novo and induced by a treatment |
| | | | (q23: q25) | |
| 30 | P300 | 22q13.2 | t (11; 22) | Very rare ANLL, induced by a treatment |
| | | | (q23; q25) | |
| 31 | RARA | 17q12 | t (11; 17) | A single case up to the present |
| | | | (q23; q12) | |
| 32 | SEPTIN | Xq22 | t (X; 11) | A single case of AML in an infant. |
| | | | (q22; q23) | |
| 33 | MLL-DUP | 11q23 | | ANLL, MDS M1, M2 and M4 ANLL myeloid lineage; |
| | | | | secondary leukemia |
| | | | | Acute myelodisplastic syndrome |

In this exemplary realization of the analytical process of the invention the probes intended for hybridization are assembled in the surface of the support in such a manner that the revation of the hybridization products on these probes causes the structure of the MLL gene to appear and the rearrangement identified in a position permits an immediate and direct visual reading of the result.

The analytical support also comprises controls of the colorimetric reaction, the efficacy of the reaction and the orientation of the biochip.

As regards the control of the colorimetric reaction, a third set of polynucleotide probes specific to a nucleotide sequence with no relationship to the nucleotide sequences to be analyzed is assembled, e.g., on a distinct zone of the support in a particular geometric configuration, e.g., in the form of an inverted “L” relative to the second set of probes. This shape can also serve as a positioning mark for the support.

As concerns a negative control, one or several polynucleotide probes representing a region situated upstream from the meeting points of each of the partner genes described (first one of each line) can also be arranged on the support. If signals appear on these controls, they can be indicators of a “false” reaction.

Each partner gene is represented on the support by a series of oligonucleotide probes selected to describe the known partner genes to be involved in translocation events by truncating the 300 base pairs.

This arrangement is advantageously selected to permit a more precise localization at the molecular level, for the positive samples, of the meeting points for each partner gene according to the appearance or non-appearance of a signal on the majority of the 5′ sequences, identifying the corresponding hybridization products. In the case of a positive sample, several spots close to the meeting point should have a detectable signal. If a single spot presents a detectable signal, this can indicate an erroneous reaction.

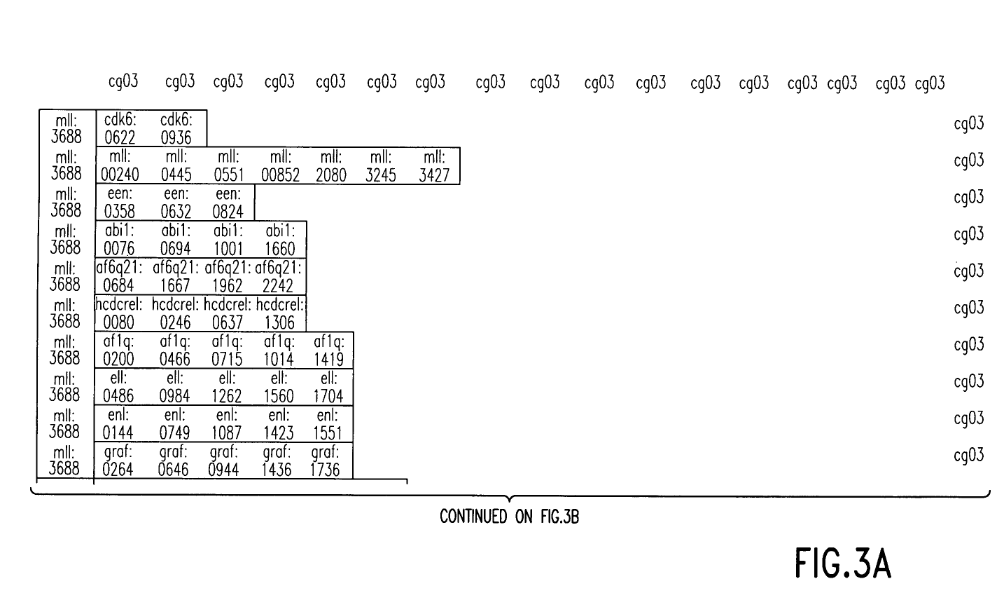

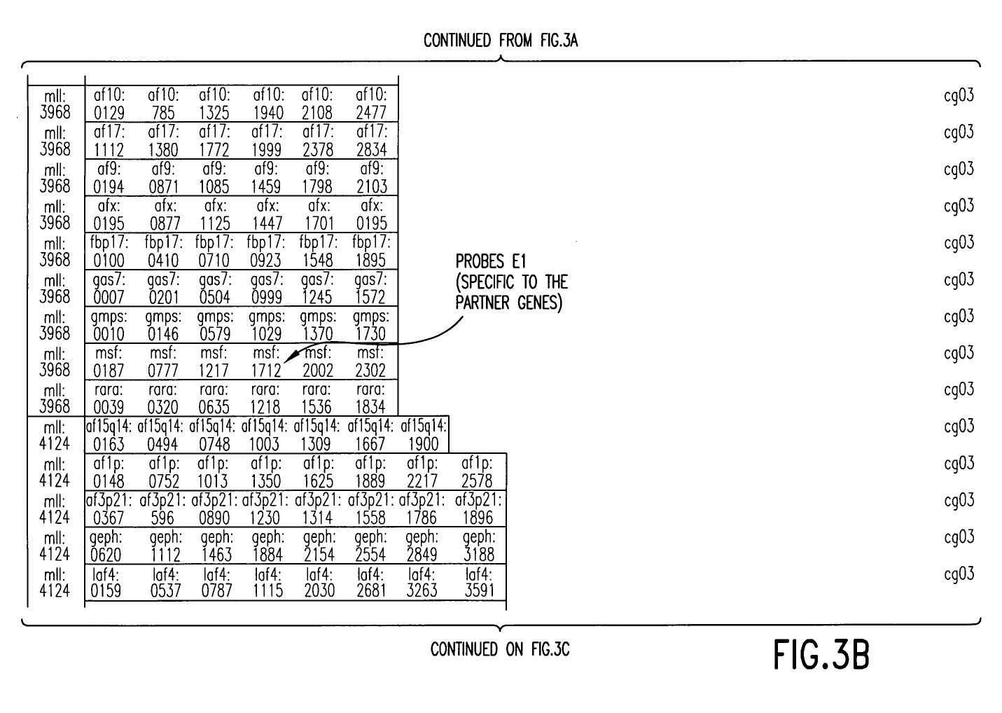

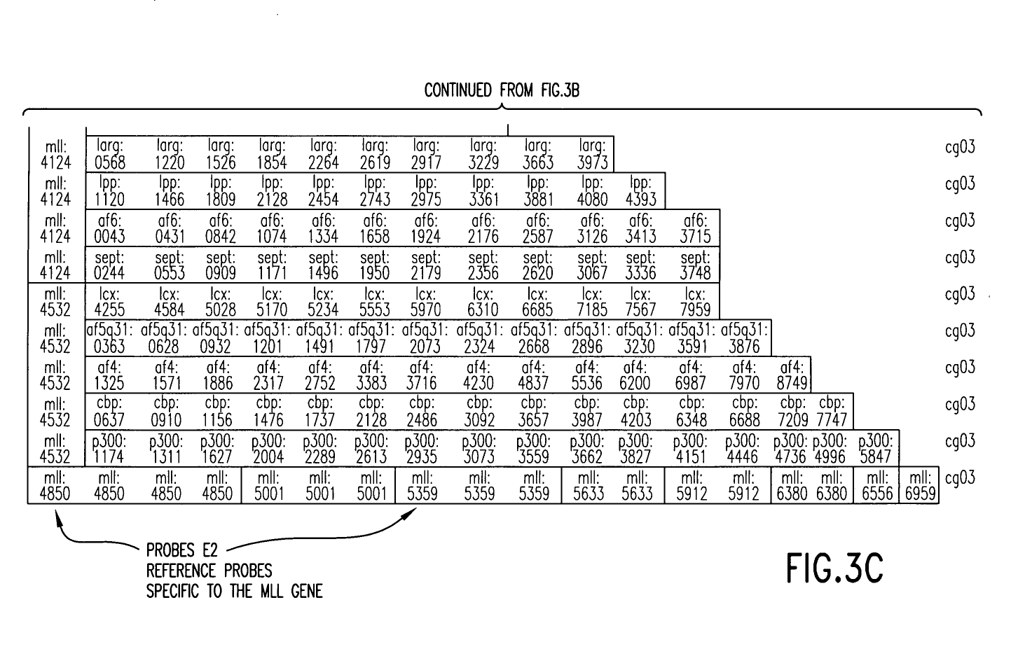

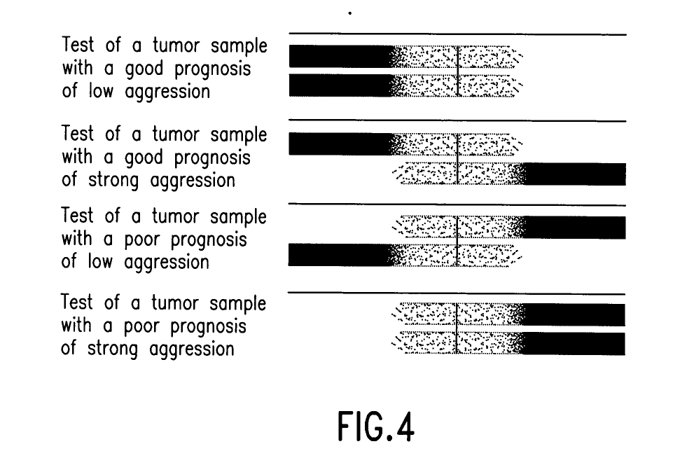

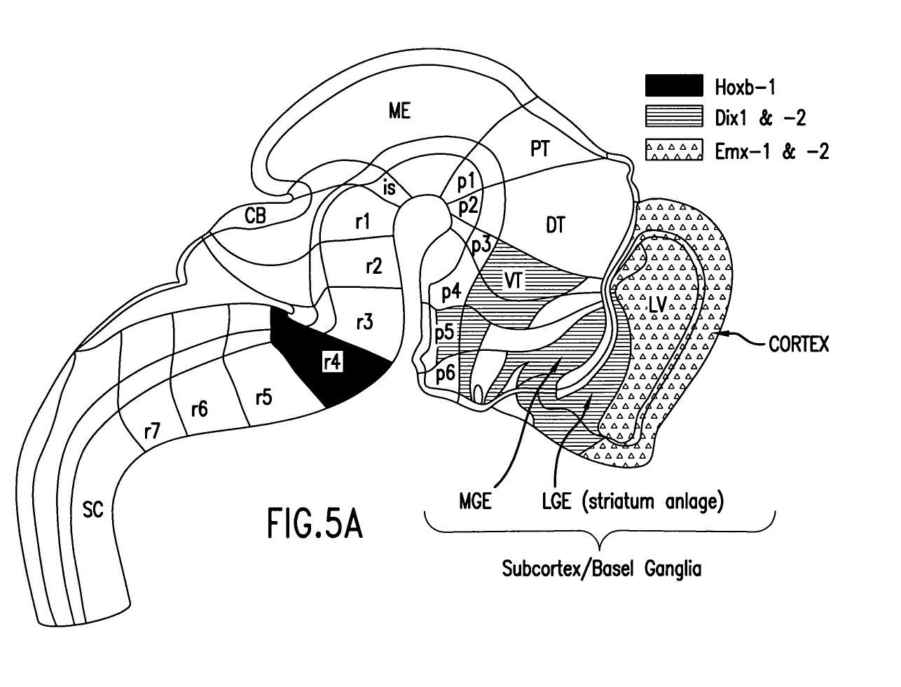

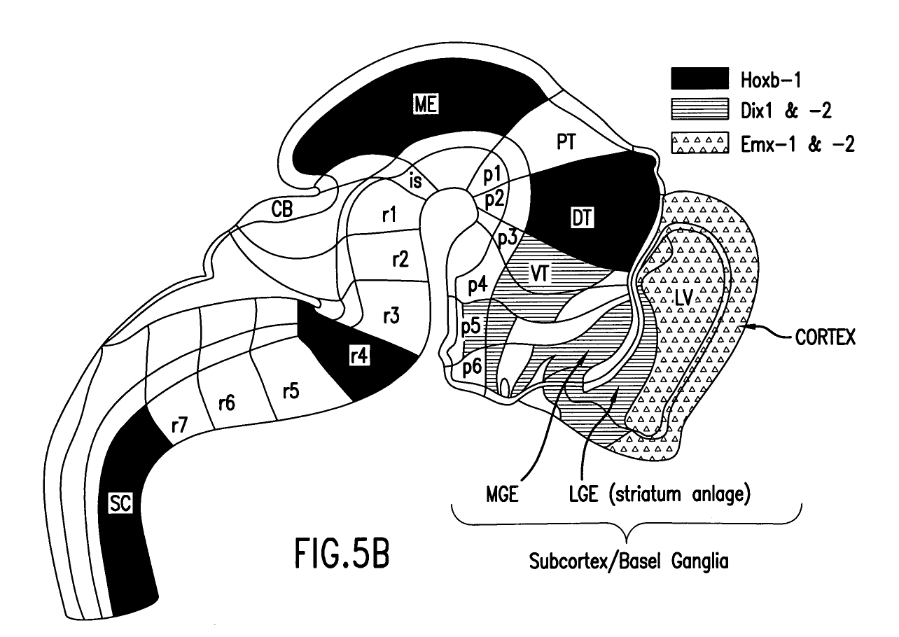

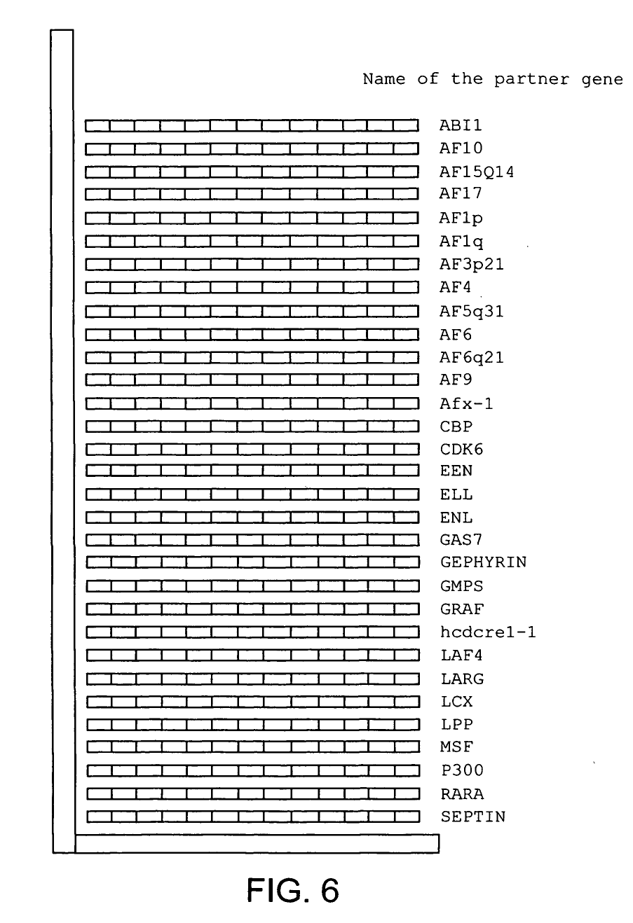

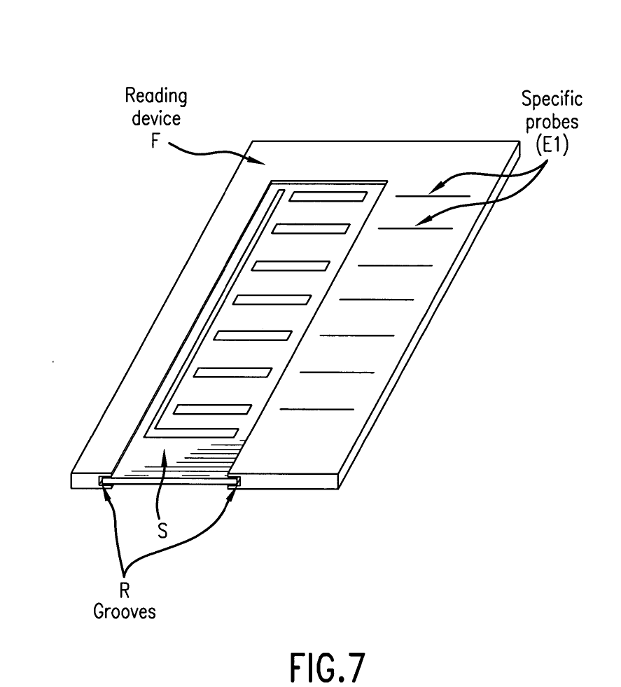

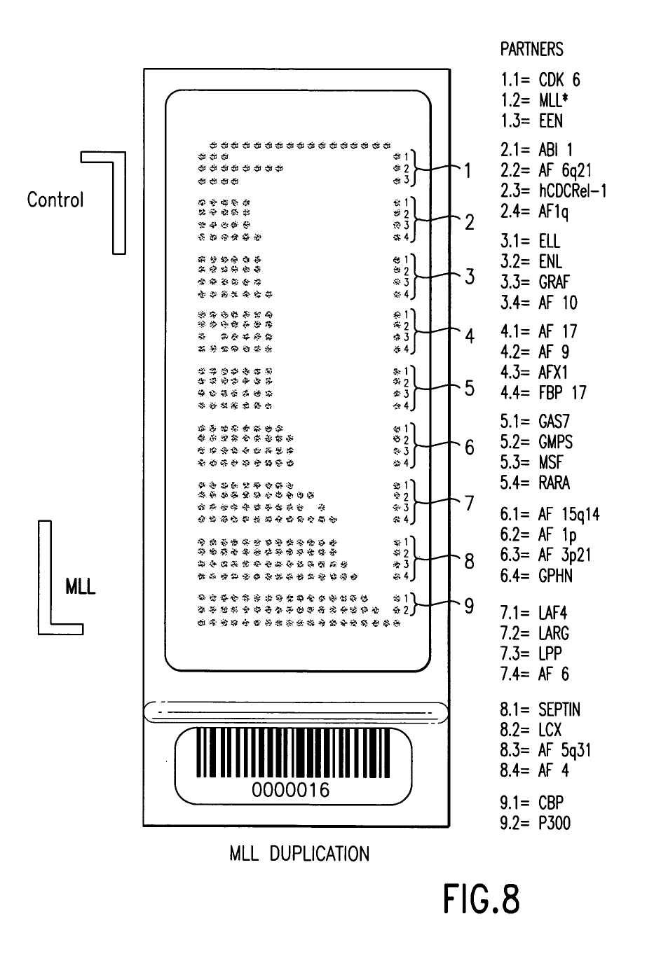

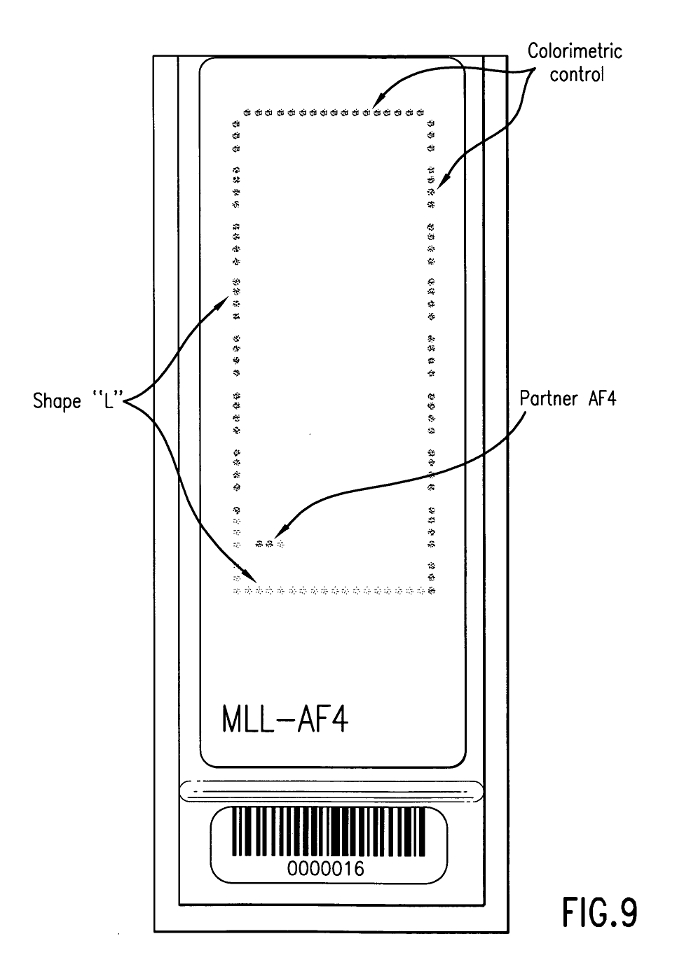

FIG. 3 shows one preferred arrangement of the probe sets on the support, including the control probes.

The second set of reference probes (E2) corresponding to specific sequences of the MLL gene is assembled on the first vertical column on the left and the first horizontal line at the bottom. This arrangement in a right “L” controls positively the efficacy of the reaction.

The first set (E1) of the specific probes of each of the thirty-two partner genes is arranged on the thirty-two other horizontal lines.

A set of marked probes, control for the calorimetric reaction, is arranged in the last column on the right and the top horizontal line.

### Example 2

Analysis Of The Levels Of Gene Expression And Prognosis Applied To Mammary Tumors

The study of the expression of genes on a large scale on a DNA chip has allowed the selection of groups of marker genes for which there are correlations between the quantity of MRNA present in the tumor and the clinical development of the patients. The demonstration on a diagnostic chip of the overexpression or of the underexpression of these groups of genes allows an indication of the seriousness of the disease and the therapy to be given. Thus, if the group of marker genes of a good diagnosis (BD) is underexpressed whereas the group of marker genes of a poor diagnosis (MD) is overexpressed, one can then decide, e.g., to use a treatment based on hormones or to use adjuvant chemotherapy.

In this exemplary realization of the analytical process of the invention the probes are assembled on the surface of the support in such a manner that the display of the hybridization products on them clearly shows the groups of genes as a function of the expected response: The BD marker genes are grouped, e.g., on the right of the support and the MD markers on the right. The control genes are arranged on the left (FIG. 2).

The intensity of the signals obtained for each group permits a classification by direct visualization with the naked eye of the analyzed tumor.

### EXAMPLE 3

Analysis Of The Levels Of Gene Expression Involved In The Positioning Of Organs During The Course Of Development

The study of the expression of genes on a large scale on a DNA chip has allowed the selection of groups of marker genes for which there are correlations between the quantity of mRNA present in the stem cell and its space-time development as a cell forming adult tissue. The demonstration on a DNA chip of the overexpression or underexpression of these groups of genes permits positioning of the cells to be specified during their developments.

In this exemplary embodiment of the analytical process of the invention the probes are assembled on the surface of the support in such a manner that the display of the hybridization products formed on these probes clearly shows the groups of genes as a function of their position in the adult tissue. The intensity of the signals obtained for each group permits an immediate visual classification of the analyzed stem cells.

1. A process for analyzing a complex biological state of a biosystem from a sample of nucleic acids isolated from the biosystem comprising hybridization of the nucleic acids with probes fixed on a support, comprising:
(a) contacting marked polynucleotide sequences prepared from nucleic acids of the sample with a first set of specific polynucleotide probes (E1) of a biological state and at least a second set of reference polynucleotide probes (E2) prepared from a molecule of reference nucleic acid for the biological state under conditions permitting the formation of hybridization products, and
(b) detecting the hybridization products formed in step (a), wherein the first and second sets (E1, E2) are fixed on a support and the first set of probes comprises n groups of probes, each group being characteristic of a different genetic profile that influences the biological state and can therefore alter it, and each group being assembled on the support in separate positions that are also separated from that (those) of the second set (E2) of probes to permit analysis of the genetic profile of the biological state by detecting the position of the hybridization products formed with at least one probe of the first set, relative to the position of hybridization products formed with the second set of probes.

2. The process according to claim 1, wherein the biological state is a physiological state of a patient and the physiological state is analyzed from a sample of nucleic acids of the patient,
the first set of probes (E1) is a set of polynucleotide probes prepared from specific molecules of nucleic acid of the physiological state and
the second set of reference probes (E2) is a set of probes prepared from a molecule of reference nucleic acid for the physiological state under conditions permitting formation of hybridization products.

3. The process according to claim 1, wherein the polynucleotide sequences of step (a) are marked by any type of marker permitting detection of the position of the hybridization products of step (b) with the naked eye.

4. The process according to claim 1, wherein each of the reference polynucleotide probes is different and prepared from a molecule of nucleic acid corresponding structurally or functionally to a genetic profile of which each of the n groups of probes of the first set is characteristic.

5. The process according to claim 1, wherein the reference polynucleotide probes are different and/or identical and prepared from a molecule of reference nucleic acid for the biological state analyzed.

6. The process according to claim 1, wherein each of the n groups of probes of the first set is arranged in a geometric configuration relative to at least one probe of the second set.

7. The process according to claim 1, wherein positions of each of the n groups of probes of the first set is in alignment with or around at least one probe of the second set.

8. The process according to claim 1, wherein the probes of the second set are aligned and the probes of each of the n groups of the first set are in alignment with a probe of the first set in such a manner that the n alignments of the probes of the first set are substantially perpendicular to the alignment of the probes of the second set.

9. The process according to claim 1, wherein step (a) comprises implementing a second second set of reference polynucleotide probes.

10. The process according to claim 1, wherein the first set of probes (E1) comprises a plurality of probes comprising at least one probe capable of separately hybridizing a nucleic acid sequence of one or several partner genes selected from the group consisting of AB11; AF10; AF15q14; AF17; 1F1p; AF1q; AF3p21; AF4; AF5q31; AF6; AF6q21; AF9; AFX-1; CPB; CDK6; EEN; EEL; ENL; FBP17; GAS7; GEPHYRIN; GMPS; GRAF; hcdcrel-1; LAF4; LARG; LCX; LPP; MSF; p300; RARA and SEPTIN and the second set of probes (E2) comprises a plurality of probes capable of hybridizing all or part of a specific sequence of nucleic acid of the MLL gene.

11. The process according to claim 9, wherein the second second set of reference polynucleotide probes is identical to the reference probes of the first set and that the probes of the second set are in positions separated from the other probe sets and in a geometric configuration relative to them.

12. The process according to claim 9, wherein the probes of the first second set are aligned substantially perpendicularly to the end of the alignment of the probes of the second second set in such a manner as to form a geometric configuration in the shape of an “L”.

13. The process according to claim 1, wherein visual detection of hybridization positions is carried out with a mask comprising elements of visual reference marks for each set of probes (E1, E2) and control probes.

14. The process according to claim 13, wherein the mask comprises a window in the shape of an “L” that can be superposed on the second set of probes aligned to form a geometric configuration in the form of an “L” on the support and n windows substantially perpendicular to one of the arms of the “L”, each of which n substantially perpendicular windows can be superposed on each of the n alignments of the probes of the first set substantially perpendicular to the alignment of the probes of the second set.

15. The process according to claim 13, wherein the mask comprises elements of visual reference marking for each set of probes (E1, E2) and has a geometric configuration reproducing a histological configuration of a tissue or an organ of the system to be analyzed.

16. A support comprising a first set of polynucleotide probes (E1) fixed to a surface of the support and at least one second set of polynucleotide probes (E2) arranged according to claim 1.

17. The support according to claim 16, comprising hybridization products of marked polynucleotide sequences on the surface prepared from nucleic acids of the sample with a part of the probes of the first set and all the probes of the second set to form a geometric figure on the surface of this support that can be detected by the naked eye.

18. The support according to claim 17, wherein the geometric figure comprises an L formed by the hybridization products of marked polynucleotide sequences prepared from nucleic acids of the sample with the probes of the second set.

19. A process for directly visualizing a molecular hybridization reaction comprising carrying out the analytical process according to claim 1 and viewing the hybridization product obtained.

20. A device for reading a support for carrying out the analytical process according to claim 1, comprising a fixed element (F) comprising inscriptions relative to a first set of specific probes (E1) that permit identification on the support of positioning of hybridization products with the specific probes and a cavity with a shape and size similar to those of the support in which cavity the support is arranged by sliding in grooves (R) provided at an end thereof.

21. A process for following up a therapeutic treatment of a patient comprising performing successive analyses of the patient's biological state according to the analytical process of claim 1 at different stages of the therapeutic treatment.

22. A process of in-vitro detecting the MLL gene in a patient comprising performing the process of claim 10 and viewing the results.
